(19) United States
(12) Patent Application Publication
(10) Pub. No.: US 2005/0124878 A1
(43) Pub. Date: Jun. 9, 2005

(54) Methods and apparatus for objective fetal diagnosis

(76) Inventor: Reuven Sharony, Kfar Saba (IL)

Correspondence Address: NATH & ASSOCIATES, 1030 15th STREET, NW, 6TH FLOOR, WASHINGTON, DC, 20005, US

(21) Appl. No.: 10/482,295

(22) PCT Filed:
(86) PCT No.: PCT/IL02/00542

(30) Foreign Application Priority Data
Jul. 2, 2001 (IL) 144110

(51) Int. Cl. A61B 8/00

(52) U.S. Cl. 600/437

(57) ABSTRACT

Fetal diagnostic apparatus (10) which comprises ultrasonic imaging apparatus (12) for producing ultrasonic images, (14) the images comprises a multiplicity of pixels (16); an ultrasonic transducer (18) that can be placed upon a patient, in data communication with the ultrasonic imaging apparatus (12); and a processor (22) in data communication with the ultrasonic imaging apparatus that measures changes in the pixels (16) with respect to time.

### FIELD OF THE INVENTION

The present invention relates generally to non-invasive diagnostic methods and apparatus, and particularly to improved methods and apparatus for objective fetal diagnosis.

### BACKGROUND OF THE INVENTION

Ultrasound has become a commonplace and routine method for non-invasive diagnosis of well-being of a fetus and progress of pregnancy. Ultrasound is used to check and monitor fetal growth, “breathing” (i.e., diaphragm movement) and limb movement, for example.

Some of the parameters detectable with ultrasound are quantities that can be measured, categorized and repeated with the same general degree of accuracy. For example, size of a fetal limb can be measured and compared with the size of a “normal” limb, i.e., the limb size of a majority of a representative fetal population. The limb size measured by one practitioner will generally match the size measured by another practitioner, thereby providing an acceptable and repeatable parameter for fetal monitoring and diagnosis.

However, fetal movement, such as that of the diaphragm or limbs, remains a subjective, rather than objective, test, and all the more so in borderline cases. Although some practitioners may claim proficiency in interpreting observations of fetal movement, nevertheless it has been found that such interpretations can vary significantly between practitioners, and can be inaccurate and even misleading. In one extreme example, active arm and leg movement can be interpreted by one practitioner as being indicative of a healthy, lively and active fetus. However, it is possible that in reality the active arm and leg movement is due to the umbilical cord wrapped around the neck of the fetus. The fetus is in distress, writhing in pain, and the supposedly healthy limb movement is actually indicative of danger. As another example, it is sometimes difficult for a practitioner to observe several fetal movements at the same time. The practitioner may be concentrating on heart movement, for example, and ignoring hand or feet movement. Clearly the prior art is problematic and an objective, ultrasonic, reproducible and automatic, fetal diagnostic method is needed.

### SUMMARY OF THE INVENTION

The present invention seeks to provide novel methods and apparatus for objective, reproducible and automatic fetal diagnosis. The present invention exploits the fact that an ultrasonic image comprises a multiplicity of pixels. The invention quantifies fetal movement by measuring changes in the pixels with respect to time. The pixels are taken from a representative area of the ultrasonic image, either the whole image or a “zoom” of a particular region of interest, such as the diaphragm. The apparatus of the invention can conveniently visually and/or audibly display (or plot) the pixel changes, such that any practitioner can easily and objectively judge total or local fetal movement, as desired. The practitioner can study and judge the fetal well-being either during or after the ultrasonic monitoring. Full documentation of the plots and pixel changes is provided for future reference.

Moreover, it is herein postulated that despite the wide variety of reasons for fetal movement, which range from healthy reasons to dangerous reasons as mentioned in the background, nonetheless a plot of the changes in the patterns of a representative portion of fetal ultrasonic images with respect to time, generally follows a normal distribution curve of pattern changes associated with fetal movement of a large representative fetal population. The system of the invention acquires data regarding the time change of the patterns of fetal movement over a predetermined period of time, and determines in which range of the normal distribution the data lie. It is postulated that the time change of the patterns associated with abnormal, unhealthy fetal movement (either overactive or underactive movement of a fetus, each being associated with different prenatal problems) lies in the asymptotic regions of the normal distribution, i.e., beyond the 2σ or 3σ limits of the normal distribution. In contrast, the time change of the patterns associated with normal, healthy fetal movement lies within the majority of the area under the normal distribution curve, i.e., within the 2σ or 3σ limits of the normal distribution. Thus, by monitoring the time-dependent change of patterns of fetal movement, one can objectively associate fetal movement with fetal health, condition and state.

In addition to the above ultrasonic diagnostic tool, the present invention provides another non-invasive method for indicating a high risk for the fetus having Down's syndrome. The inventor has surprisingly found that administration of a certain range of dosage of atropine to pregnant women, can cause tachycardia in fetuses with Down's syndrome, whereas the same dosage does not generally change heartbeat rate in normal fetuses to the same extent.

There is thus provided in accordance with a preferred embodiment of the present invention fetal diagnostic apparatus including ultrasonic imaging apparatus for producing ultrasonic images, the images including a multiplicity of pixels, an ultrasonic transducer that can be placed upon a patient, in data communication with the ultrasonic imaging apparatus, and a processor in data communication with the ultrasonic imaging apparatus that measures changes in the pixels with respect to time.

In accordance with a preferred embodiment of the present invention a display is in data communication with the processor, which displays the changes in the pixels with respect to time. The display may be visual or audible.

There is also provided in accordance with a preferred embodiment of the present invention a method for diagnosing a fetus inside a pregnant woman, including acquiring fetal ultrasonic images, the images including a multiplicity of pixels, measuring changes in the pixels of a representative portion of the fetal ultrasonic images with respect to time, over a predetermined period of time, the changes in the pixels being associated with a pattern of fetal movements, and monitoring changes in the pattern of the fetal movements with respect to time. The method also preferably includes displaying the changes in the patterns of fetal movements with respect to time.

In accordance with a preferred embodiment of the present invention the method further includes choosing a particular region of interest of the fetus, and tracking pixel changes only in the particular region of interest.

Further in accordance with a preferred embodiment of the present invention an ultrasonic transducer is used to acquire the fetal ultrasonic images in a viewing window, and movement of the viewing window is controlled such that the particular region of interest is generally continuously in the viewing window.

In accordance with a preferred embodiment of the present invention the method further includes providing a normal distribution curve of changes associated with patterns of fetal movement of a large representative fetal population, determining in which range of the normal distribution the measured changes of patterns of fetal movements lie, and diagnosing the fetal movements based on the range of the normal distribution in which the measured changes of patterns of fetal movements lie.

Further in accordance with a preferred embodiment of the present invention there is provided a method for diagnosing a fetus inside a pregnant woman for a risk of having Down's syndrome. The method comprises administering to the pregnant woman a cholinergic signaling inhibitor; and monitoring fetal heartbeat rate thereafter; whereby if the fetal heartbeat rate accelerates beyond a predetermined threshold, then the fetus is considered to have an increased risk of Down's syndrome.

Further in accordance with a preferred embodiment of the present invention about 0.6-1.2 mg of atropine are administered to the pregnant woman, and the fetal heartbeat rate is monitored thereafter. If the fetal heartbeat rate accelerates beyond a predetermined threshold, then the fetus is considered to have an increased risk of Down's syndrome.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification will control.

Implementation of the method and apparatus of the present invention involves performing or completing selected tasks or steps manually, automatically, or a combination thereof. Moreover, according to actual instrumentation and equipment of preferred embodiments of the method and apparatus of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions. The apparatus and method of the present invention are hence readily convertible into a telemedicine operation format.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a simplified pictorial illustration of fetal diagnosis apparatus constructed and operative in accordance with a preferred embodiment of the present invention;

FIGS. 2 and 3 are simplified pictorial illustrations of a display of the apparatus of FIG. 1, showing different amounts of fetal movement and the different changes of pixels over time associated with these movements;

FIG. 4A is a simplified graphical illustration of two examples of changes of patterns of fetal movements with respect to time for a fetus of a given age;

FIG. 4B is a simplified graphical illustration of a normal distribution curve of pattern changes associated with fetal movement of a large representative fetal population; and

FIG. 5 is a simplified pictorial illustration of a fetal diagnosis method in accordance with another preferred embodiment of the present invention, wherein atropine is administered to a pregnant woman and fetal heartbeat rate is measured.

### DETAILED DESCRIPTION OF PREFERRED EMBODIMENTS

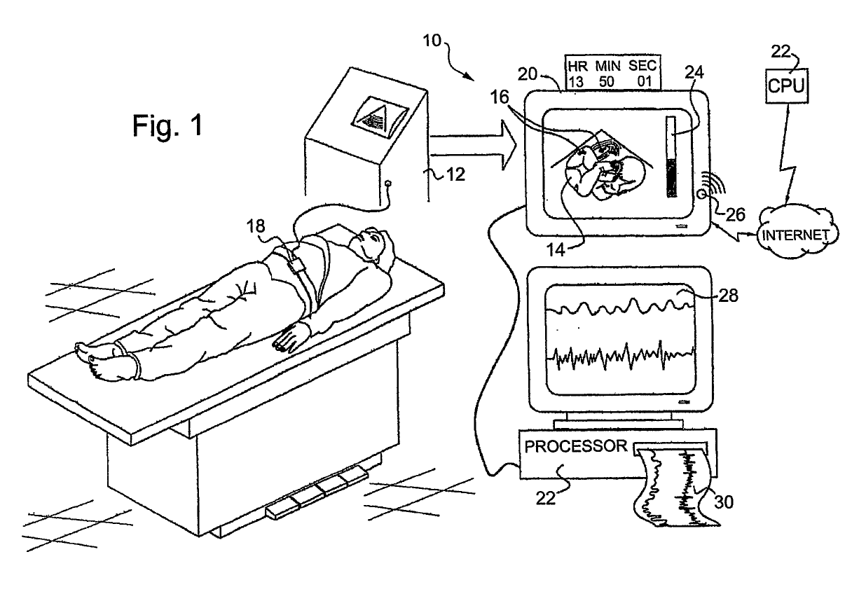

Reference is now made to FIG. 1 which illustrates fetal diagnostic apparatus 10, constructed and operative in accordance with a preferred embodiment of the present invention. Apparatus 10 preferably includes ultrasonic imaging apparatus 12 for producing ultrasonic images 14, which comprise a multiplicity of pixels 16. Ultrasonic imaging apparatus 12 is in data communication with an ultrasonic transducer 18 that can be placed upon a patient. A monitor 20 is preferably provided for displaying the ultrasound images 14, in data communication with ultrasonic imaging apparatus 12.

In accordance with a preferred embodiment of the present invention, a processor 22 is in data communication (direct or indirect, wired or wireless) with ultrasonic imaging apparatus 12 that measures changes in the pixels 16 with respect to time. This change in the pixels is preferably displayed at a display 24 in data communication with processor 22. Display 24 is preferably a visual display, such as a bar graph displayed on the screen of monitor 20. Alternatively or additionally, there may be provided an audible display 26. Alternatively or additionally, the change in the pixels may be shown graphically on another monitor screen 28 or outputted as a printed graph 30. Processor 22 may be either local to the displays and ultrasonic imaging apparatus 12, or alternatively, may be at a remote site and connected to ultrasonic imaging apparatus 12 and the displays by means of a service provider network or Internet, for example.

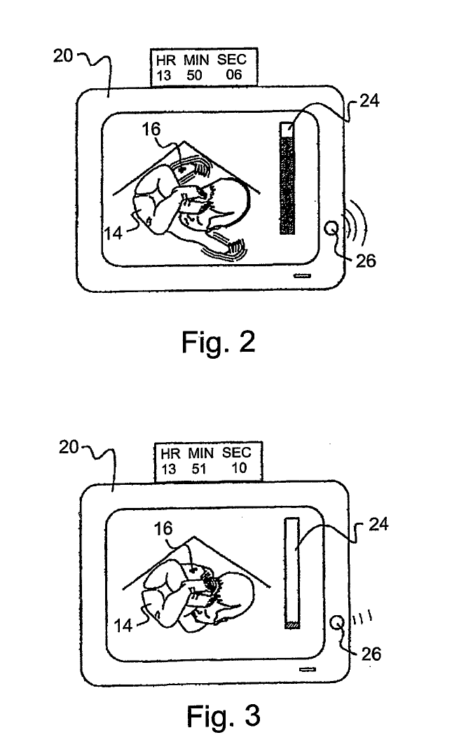

As seen in FIGS. 2 and 3, during the period of time that ultrasonic transducer 18 is on the patient, the fetal movements can become greater/faster (FIG. 2) or smaller/slower (FIG. 3). In each case, display 24 or 26 changes accordingly. For example, in FIG. 2, display 24 displays a larger bar graph. Display 26 can emit a louder sound. Conversely, in FIG. 3, display 24 displays a smaller bar graph, and display 26 can emit a quieter sound.

The pixels 16 that are monitored may be from all or part of the image 14. For example, the practitioner may choose a particular limb and processor 22 may then be commanded to monitor pixel changes only, in the region of that limb. More specifically, processor 22 may constantly track a particular limb or region of a limb. In simplistic terms, “tracking” means that ultrasonic transducer 18 “locks on” to a particular region of interest, for example, the right forearm of the fetus. This means that transducer 18 “sees” or senses the forearm in a viewing window. As long as the forearm appears in this viewing window, transducer 18 is successfully tracking the forearm. Since the forearm moves about, the viewing window of transducer 18 must also be moved accordingly in order to continuously track the forearm and not lose “sight” of the forearm. Processor 22 controls the movement of the viewing window of transducer 18 by employing methods or algorithms for single-target or multi-target tracking, which are well known in the art of radar tracking. (Radar tracking methods are discussed in many texts, for example, George W. Stimson, “Introduction to Air-borne Radar”, Hughes Aircraft Company, p. 472-476.)

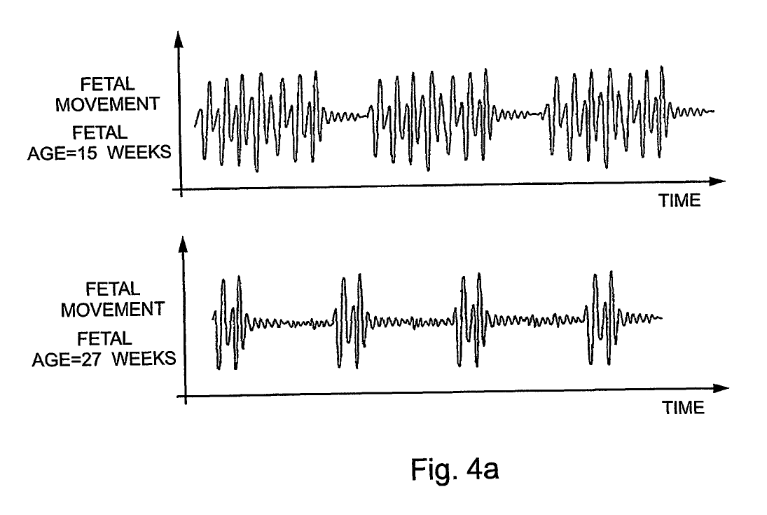

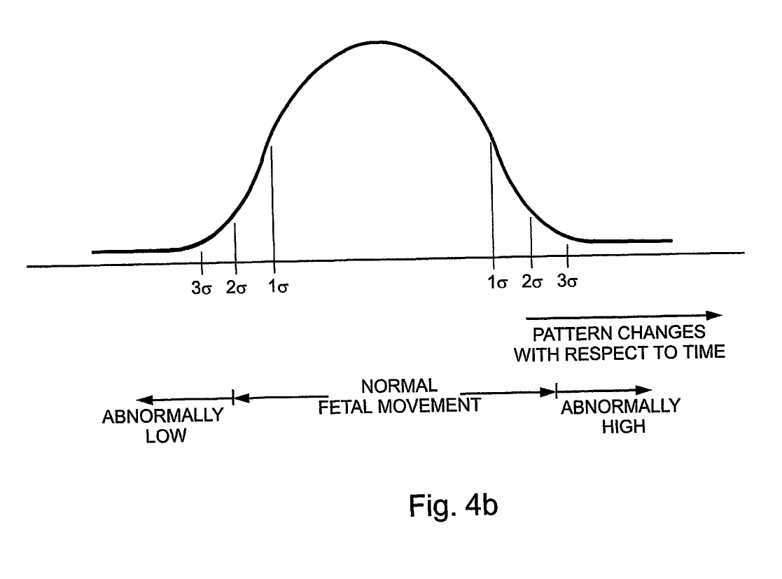

The pixel changes of the particular region of interest (in the above example, the right forearm) are then monitored with respect to time, as described hereinabove, the change in the pixels being preferably displayed at display 24. The fetal movements associated with the pixel changes form a pattern which changes with time. FIG. 4A illustrates two examples of changes of patterns of fetal movements with respect to time for a fetus of a given age. The present invention provides an objective evaluation of the changes of patterns of fetal movements with respect to time for a fetus of a given age, as is now explained.

It is postulated that a plot of the changes of patterns of fetal movements with respect to time for a fetus of a given age, generally follows a normal distribution curve of changes associated with fetal movement of a large representative fetal population, as seen in FIG. 4B. Processor 22 determines in which range of the normal distribution the data lie. It is postulated that the time change of patterns associated with abnormal, unhealthy fetal movement (either overactive or underactive movement of a fetus, each being associated with different prenatal problems) lies in the asymptotic regions of the normal distribution, i.e., beyond the 2σ or 3σ limits of the normal distribution. In contrast, the time change of patterns associated with normal, healthy fetal movement lies within the majority of the area under the normal distribution curve, i.e., within the 2σ or 3σ limits of the normal distribution. Thus, by monitoring the time-dependent change of patterns, such as those shown in FIG. 4A, one can objectively associate fetal movement with fetal health, state and condition.

It is noted that the same healthy fetus can have different patterns of movement depending on the age. Each time the fetus is monitored, the patterns of fetal movement are preferably recorded and documented for future reference. The progress of the fetus can be judged by studying the patterns recorded in accordance with the present invention, as described hereinabove.

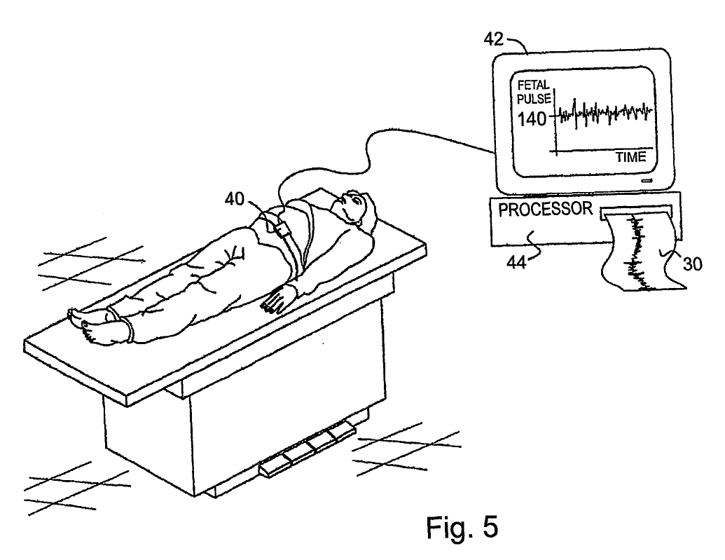

Reference is now made to FIG. 5 which illustrates a fetal diagnosis method in accordance with another preferred embodiment of the present invention. In this method, about 0.6-1.2 mg of atropine, or a functionally equivalent amount of other, preferably reversible, cholinergic signaling inhibitors, such as, acetylcholinesterase inhibitors, e.g., physostigmine, pyridostigmine, neostigmine and edrophonium, and/or acetylcholine receptor (muscarinic (M1 or M2) or nicotinic) inhibitors (antagonists), e.g., scopolamine, trimethapan, tetraethylammonium, mecamylamine, benztropine (antimuscarinic, especially the phenothiazine (Thorazine) group of antipsychotic medications and the tricyclic (Elavil) group of antidepressants), and pirenzepine (appears to be selective for M1 receptors), is administered to a pregnant woman, preferably by means of a suppository, skin patch, tablet or the like, and the fetal heartbeat rate is monitored, preferably by means of a fetal heartbeat rate sensor 40. The fetal heartbeat rate is displayed on a monitor 42. A processor 44 may be provided for processing data received from fetal heartbeat rate sensor 40. Any method applicable for monitoring fetal heart rate is useful in context of this aspect of the present invention, such methods include, but are not limited to, use of a stethoscope, Doppler ultrasound and the method of the present invention, described herein in context of FIGS. 1-4.

The ontogeny of muscarinic cholinergic receptors in developing human brain is well know. It was analyzed by in vitro receptor autoradiography with [3H]Quinuclidinyl Benzilate. It was found that muscarinic receptors develop relatively early; the levels at 24 weeks of gestation were comparable or even higher then the values in the adult brain, and that the levels of both M1 and M2 receptors increase with age. M1 receptors are concentrated mainly in forebrain regions while M2 receptors dominated in the thalamus. Scatchard analysis revealed Kd and Bmax values which are comparable to the adult values. Brains of aborted Down's syndrome fetuses were also examined. These brains exhibit comparable levels and similar distribution to normal non-Down fetuses except for a modest increase of receptor levels which was observed in the striatum (Bar-Peled O, Israeli M, Ben-Hur H, Hoskins I, Groner Y, Biegon A. Developmental pattern of muscarinic receptors in normal and Down's syndrome fetal brain—an autoradiographic study. Neurosci Lett 1991 Dec. 9;133(2):154-8).

The mydriatic response to eye drops of the anticholinergic agent tropicamide at very low concentration (0.01%) has been studied in people with Down's syndrome. By comparison with healthy subjects people with Down's syndrome had responses approximately three times greater, suggesting a peripheral imbalance between cholinergic and adrenergic autonomic influences (Sacks B, Smith S. People with Down's syndrome can be distinguished on the basis of cholinergic dysfunction. J Neurol Neurosurg Psychiatry 1989 November;52(11):1294-5).

Atropine is a well known substance used for various medical purposes, such as preanesthetic medication or as an ingredient in spasmolytic suppositories administered to treat contractions in pregnant women. The effects of atropine on children or adult subjects with Down's syndrome have been published in the medical literature. J. M. Berg et al., “Atropine in Mongolism”, Lancet 2:441-442, September 1959, reports that atropine placed in the conjunctival sac of a person with Down's syndrome, causes abnormally great mydriasis (dilation of the pupil). The cause of the reaction is not known and has been attributed to a structural anomaly present in 95% of Down's syndrome patients, the anomaly being hypoplasia (i.e., incomplete development) of the peripheral stroma of the iris.

W. S. Harris and R. M. Goodman, “Hyper-Reactivity to Atropine in Down's Syndrome”, The New England Journal of Medicine, 8:407-410, Aug. 22, 1968, suggests that Down's syndrome patients have a pharmacogenetic abnormality that increases sensitivity to atropine. It is known that a small dose, such as 0.24 mg, of atropine sulfate has a bradycardiac effect (i.e., decelerates the heartbeat rate), whereas a large dose, such as 2 mg, has a tachycardiac effect (i.e., accelerates the heartbeat rate). Harris and Goodman report that some patients with Down's syndrome have an abnormally sensitive tachycardiac reaction to atropine. More specifically, quoting from page 409, fourth paragraph of the discussion, “Clearly, young adult, white, male patients with mongolism have an increase sensitivity to the cardioacceleratory effects of atropine. The effects of atropine in patients who are female, Negro, or of a different age group remain to be determined.” Several possible explanations are offered for the phenomenon, but as concluded in the last paragraph on page 409, the “mechanism is unclear”.

Thus, it is not clear at all from Harris and Goodman what the effects of atropine would be on fetal heartbeat rates. The present invention provides the missing answer. In accordance with a preferred embodiment of the present invention, administering about 0.6-1.2 mg of atropine to a pregnant woman has a tachycardiac effect on fetal heartbeat rate. In other words, this dosage of atropine administered to a fetus increases the heartbeat rate above the normal range expected for a fetus of the same stage of fetal development. For example, a fetus that has reached 17 weeks of development has a pulse rate of about 150 beats per minute before administration of atropine to the mother. Administration of the above dosage of atropine will significantly raise the pulse rate, e.g., to at least 167 beats per minute. The same dosage does not generally change heartbeat rate in normal fetuses to the same extent. Accordingly, if the fetal heartbeat rate accelerates beyond a predetermined threshold, then the fetus is considered to have an increased risk of Down's syndrome. The method of the present invention may be used in conjunction with other diagnostic tests, such as the ultrasonic method described hereinabove.

Telemedicine is a fast growing field in which medical data and/or records are networked, typically in real time, to remote center via a network for purposes or archiving and/or analysis. Telemedicine has the advantages of allowing non experts to use medical instrumentation in diagnosis and have experts or sophisticated diagnosing software analyze the results and report of their analysis to the non expert in real time. In many cases telemedicine takes the advantages of the Internet (www) as the network through which medical data is networked to the remote center and back. The methods and apparatus described herein are suitable for telemedicine applications.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. Fetal diagnostic apparatus comprising:
ultrasonic imaging apparatus for producing ultrasonic images, said images comprising a multiplicity of pixels;
an ultrasonic transducer that can be placed upon a patient, in data communication with said ultrasonic imaging apparatus; and
a processor in data communication with said ultrasonic imaging apparatus that measures changes in the pixels with respect to time.

2. Apparatus according to claim 1 and further comprising a display in data communication with said processor that displays the changes in the pixels with respect to time.

3. Apparatus according to claim 2 wherein said display comprises a visual display.

4. Apparatus according to claim 2 wherein said display comprises an audible display.

5. A method for diagnosing a fetus inside a pregnant woman, comprising:
acquiring fetal ultrasonic images, said images comprising a multiplicity of pixels;
measuring changes in the pixels of a representative portion of said fetal ultrasonic images with respect to time, over a predetermined period of time, the changes in the pixels being associated with a pattern of fetal movements; and
monitoring changes in the pattern of the fetal movements with respect to time.

6. The method according to claim 5 and further comprising displaying the changes in the patterns of fetal movements with respect to time.

7. The method according to claim 5 and further comprising choosing a particular region of interest of the fetus, and tracking pixel changes only in said particular region of interest.

8. The method according to claim 7 wherein an ultrasonic transducer is used to acquire the fetal ultrasonic images in a viewing window, and wherein the method comprises controlling movement of the viewing window such that said particular region of interest is generally continuously in the viewing window.

9. The method according to claim 5 and further comprising:
providing a normal distribution curve of changes associated with patterns of fetal movement of a large representative fetal population;
determining in which range of the normal distribution the measured changes of patterns of fetal movements lie; and
diagnosing said fetal movements based on the range of the normal distribution in which the measured changes of patterns of fetal movements lie.

10. The method according to claim 5 and further comprising administering about 0.6-1.2 mg of atropine to the pregnant woman, and monitoring fetal heartbeat rate thereafter.

11. The method according to claim 10 wherein if the fetal heartbeat rate accelerates beyond a predetermined threshold, then the fetus is considered to have an increased risk of Down's syndrome.

12. A method for diagnosing a fetus inside a pregnant woman, comprising:
administering about 0.6-1.2 mg of atropine to the pregnant woman; and monitoring fetal heartbeat rate thereafter.

13. The method according to claim 12, wherein if the fetal heartbeat rate accelerates beyond a predetermined threshold, then the fetus is considered to have an increased risk of Down's syndrome.

14. A method for diagnosing a fetus inside a pregnant woman for a risk of having Down's syndrome, comprising:
administering to the pregnant woman a cholinergic signaling inhibitor; and
monitoring fetal heartbeat rate thereafter;
whereby if the fetal heartbeat rate accelerates beyond a predetermined threshold, then the fetus is considered to have an increased risk of Down's syndrome.

15. The method according to claim 6 and further comprising administering about 0.6-1.2 mg of atropine to the pregnant woman, and monitoring fetal heartbeat rate thereafter.

16. The method according to claim 7 and further comprising administering about 0.6-1.2 mg of atropine to the pregnant woman, and monitoring fetal heartbeat rate thereafter.

17. The method according to claim 8 and further comprising administering about 0.6-1.2 mg of atropine to the pregnant woman, and monitoring fetal heartbeat rate thereafter.

18. The method according to claim 9 and further comprising administering about 0.6-1.2 mg of atropine to the pregnant woman, and monitoring fetal heartbeat rate thereafter.
